(19) United States
(12) Patent Application Publication
(10) Pub. No.: US 2009/0020711 A1
(43) Pub. Date: Jan. 22, 2009

(54) A PARTICLE BEAM APPLICATION DEVICE AND AN IRRADIATION DEVICE AS WELL AS A METHOD FOR GUIDING A PARTICLE BEAM

(75) Inventors: Thomas Hansmann, Heidelberg (DE); Eike Rietzel, Darmstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 12/218,724

(22) Filed: Jul. 17, 2008

Related U.S. Application Data
(60) Provisional application No. 60/961,348, filed on Jul. 20, 2007.

(30) Foreign Application Priority Data
Jul. 20, 2007 (DE) 10 2007 033 894.7

(51) Int. Cl. A61N 5/00

(52) U.S. Cl. 250/492.3

(57) ABSTRACT

The invention relates to a particle beam application device for directing a particle beam at a target volume for irradiating the target volume. A beam shaping device which can be disposed in a beam path of the particle beam shapes a cross-sectional profile of the particle beam, the beam shaping device being implemented such that, after shaping, the particle beam has a line-like cross-sectional profile. A controller varies the relative position of the particle beam and target volume during an irradiation run, the controller being implemented such that, by changing the relative position, the particle beam with the line-like cross-sectional profile can be scanned over the target volume to be irradiated. A line-like cross-sectional profile of a particle beam is formed for guiding a particle beam. A trajectory of the particle beam is variably changed in a direction parallel to the beam trajectory direction.

### CROSS REFERENCE TO RELATED APPLICATIONS

The present application claims the benefit of a provisional patent application filed on Jul. 20, 2007, and assigned application No. 60/961,348. The present application also claims the benefit of a German application No. 10 2007 033 894.7 filed Jul. 20, 2007. Both of the applications are incorporated by reference herein in their entirety.

### FIELD OF THE INVENTION

The invention relates to a particle beam application device, as is used particularly for particle beam irradiation of tissue in the context of particle therapy. The invention also relates to an irradiation device having a particle beam application device of this kind. The invention further relates to a method for guiding a particle beam, as is used in particular for particle beam irradiation of tissue.

### BACKGROUND OF THE INVENTION

Particle therapy is an established method for treating tissue, particularly tumor diseases. Irradiation methods as used in particle therapy can, however, also be employed in non-therapeutic fields. These include, for example, particle therapy research work carried out on non-living phantoms or bodies, irradiation of materials, etc. In these applications, charged particles are usually accelerated to high energies, shaped into a particle beam and directed at an object to be irradiated. The particle beam penetrates the object where it releases its energy at a defined location, resulting in destruction of the tissue present there. Protons, carbon ions but also pions, helium ions or other types of ion are generally used as particles.

After particle acceleration and beam shaping, it is necessary to direct the particle beam as accurately as possible at a target volume to be irradiated, so that the energy of the particle beam is deposited as accurately as possible on a predefined target. Various known methods for this purpose exist.

One possibility is to spread the particle beam two-dimensionally, so that its cross-section is so great that large parts of the target volume to be irradiated—or even the entire target volume—are impinged by the spread particle beam. Such a technique is also known as the scattering method. In this context, collimators are often used which are inserted in the beam path of the spread particle beam and with which the cross-sectional area of the particle beam is shaped. This enables, for example, the cross-section of the particle beam to be adapted to a target volume to be irradiated. Depth modulation, i.e. the penetration depth of the particle beam, can be achieved with the scattering method by varying the energy of the particle beam.

Another possibility is the so-called raster scan method in which a particle beam with a comparatively small cross-sectional area of a few millimeters (known as a pencil beam) can be focused onto the target volume. By deflecting the charged particle beam, different raster points in the target volume can be visited consecutively, so that the particle beam releases its energy sequentially at individual raster points in the target volume. Particle beam penetration depth into the target volume and therefore homing-in on raster points at different depths in the target volume is achieved by varying the energy of the particle beam.

### SUMMARY OF THE INVENTION

The object of the invention is to specify a particle beam application device which enables a particle beam to be applied with little scatter and good insensitivity to movement of the target volume while at the same time providing rapid irradiation of the target volume. The object of the invention is also to specify an irradiation device incorporating a particle beam application device of this kind. Another object of the invention is to specify a method for guiding a particle beam which enables a particle beam to be provided with which a target volume can be rapidly irradiated with minimal unwanted scatter.

These objects are achieved by a particle beam application device and an irradiation device as well as a method as claimed in the claims.

With the particle beam application device according to the invention, a particle beam entering the particle beam application device can be directed onto a target volume in order to irradiate said target volume. For this purpose the particle beam application device has a beam shaping device which can be disposed in the beam path of the particle beam to form a cross-sectional profile of the particle beam, the beam shaping device being designed such that the particle beam, after shaping by the beam shaping device, has a line-like cross-sectional profile.

A line-like cross-sectional profile is to be understood to mean a cross-sectional profile which has a much greater extent in one direction than in another direction, so that the cross-sectional profile appears on the whole line-like, the extent in one direction corresponding to the extent of a particle beam as used e.g. in raster scanning. For example, the extent in one direction can be a few millimeters, whereas the extent in the other direction is in the order of centimeters. This differentiates the cross-sectional profile of the particle beam from known cross-sectional profiles. In raster scanning, the cross-sectional profile of the particle beam is point-like, with the particle beam extending e.g. a few millimeters in each direction, whereas in the case of a two-dimensionally spread particle beam the cross-section of the particle beam has a significant extent in both dimensions, the precise shape of the cross-sectional area often being defined by a limiting collimator.

The particle beam application device additionally incorporates a controller for changing the relative position of the particle beam and target volume during an irradiation run, the controller being designed such that, by changing the relative position, the particle beam with the line-like cross-sectional profile can be scanned over the target volume.

The underlying idea of the invention is consequently that conventional irradiation methods such as raster scanning or scattering using a two-dimensionally spread particle beam are accompanied by disadvantages. The two-dimensional spreading of the particle beam and subsequent limiting of the particle beam using a collimator tends to produce significant scatter which reduces target volume irradiation accuracy. On the other hand, the raster scan method is prone to under- or overdosing if the target volume moves, which often occurs when irradiating human tissue.

With the particle beam application device, a particle beam can now be produced which has a line-like cross-sectional profile rather than a two-dimensional or point-like cross-sectional profile. This produces considerably less scatter than with two-dimensional spreading of the particle beam. Using the controller to vary the relative position between the particle beam and the target volume it is possible to scan the particle beam with the line-like cross-sectional profile over the target volume. The line-like cross-sectional profile makes the scanning method much less prone to over- or underdosing when irradiating a moving target volume.

The beam shaping device can be controlled by a beam shaping device controller which is implemented such that the beam shaping device interacts with the particle beam in such a way that a particle beam emerging from the beam shaping device has a line-like cross-sectional profile, the controller for varying the relative position being disposable in particular downstream of the beam shaping device in the beam direction.

In an advantageous embodiment, the beam shaping device incorporates a magnetic system with which an oscillating magnetic field can be produced. Controlled by said oscillating magnetic field, a particle beam with a point-like cross-sectional profile is guided is steered to and fro in such a way that, by oscillating the particle beam with the point-like cross-sectional profile, a particle beam with a line-like cross-sectional profile is formed, the oscillating magnetic field being oriented relative to the particle beam such that the particle beam is deflected in a direction running essentially perpendicular to the direction of the beam. The advantage of this embodiment is that a particle beam application device which can be used for raster scanning requires only minor modification in order to produce a line-like cross-sectional profile in the particle beam.

In particular, the magnetic system can be controlled by a magnetic system controller with which an offset and/or an amplitude and/or a frequency of the oscillating magnetic field can be set. This provides a simple means of manipulating and shaping the profile of the line-like cross-sectional profile. In particular, the oscillating magnetic field can be variably adjusted during an irradiation run, making it possible to alter the line-like particle beam while it is being scanned over a target volume. In this way the particle beam can be precisely adapted to suit the target volume.

In another advantageous embodiment, the beam shaping device comprises a collimator with a line-like aperture which can be inserted in the path of the particle beam. In particular, the beam shaping device incorporates a beam spreader for spreading out the particle beam, which beam spreader is disposed upstream of the collimator in the beam direction. This simple beam shaping device provides a simple means of producing a particle beam with a line-like cross-sectional profile from an in particular two-dimensionally spread particle beam.

In an advantageous further development, the collimator comprises an adjusting device for varying the geometry of the line-like aperture of the collimator. For example, the length of the line-like aperture can be variably adjusted, i.e. the length of the line-like aperture can be limited. In this way the cross-sectional profile of the particle beam can be varied in a simple manner. However, it is also possible to interrupt the line-like aperture lengthwise with the adjusting device so that toroidally shaped target volumes can also be irradiated with the particle beam, which then has a discontinuous line-like cross-sectional profile. The geometry of the line-like aperture or more specifically of the line-like cross-sectional profile can be changed in particular during an irradiation run and during scanning of the particle beam over the target volume.

In an advantageous further development the collimator is designed such that a position of the line-like aperture can be varied relative to the beam trajectory, in particular perpendicular to the beam trajectory. This variation can be controlled by the controller for changing the relative position between the particle beam and the target volume to be irradiated, thereby providing a simple means of scanning the particle beam with the line-like cross-sectional profile over the target volume by moving the line-like aperture in the beam path. This can be achieved, for example, by moving the collimator with the line-like aperture as a whole relative to the beam trajectory. However, it can also be achieved by this means that the collimator is fixed and the collimator is designed such that the line-like aperture in the collimator is moved.

In an advantageous embodiment, the particle beam application device has an electromagnetic deflecting device which is disposed in particular downstream of the beam shaping device in the beam trajectory. The electromagnetic deflecting device can deflect the particle beam passing through the electromagnetic deflecting device and scan it over the target volume to be irradiated, said electromagnetic deflecting device being controlled in particular by the controller for varying the relative position between the particle beam and the target volume to be irradiated. The advantage of an embodiment of this kind is that a known particle beam deflecting device which can be used for raster scanning only requires minor modifications.

In another variant, the controller for varying the relative position between the particle beam and the target volume to be irradiated is designed to produce a control signal for a positioning device for the target volume so that, during an irradiation run, the target volume can be moved with the positioning device such that the particle beam with the line-like cross-sectional profile is scanned over the target volume. The advantage of this variant is that scanning of the particle beam over the target volume can be achieved in a simple manner, without deflecting the particle beam itself from a notional beam center axis, the particle beam application device being able to also accommodate the positioning device for the target volume.

The particle beam application device can additionally have at least one dipole magnet downstream of the beam shaping device in the beam trajectory for de-scattering the particle beam. The advantage of this variant is that particularly in the case of an inserted beam spreader for spreading the particle beam and/or a collimator disposed in the beam path of the particle beam, the beam quality is improved. In these cases, non-negligible scatter is produced which is characterized in that its mass-to-charge ratio and/or its energy is at variance with the desired characteristics of the particle beam. If the particle beam is now passed through the dipole magnet, the particle beam components having a different energy or a different mass-to-charge ratio are deflected differently in the dipole magnet compared to the particle beam components having the desired energy or mass-to-charge ratio. This enables the particle beam to be cleared of unwanted components, thereby improving irradiation quality.

In particular, the particle beam application device can have a second collimator with a line-like aperture disposed in the beam trajectory downstream of the dipole magnet. A second collimator of this kind provides a simple means of extracting the wanted components of the particle beam, as they are no longer incident through the line-like aperture of the second collimator due to the different deflection in dipole magnets.

In an advantageous variant, the second collimator is designed such that a position of the line-like aperture of the second collimator can be varied relative to the beam trajectory. In this way the second collimator or rather the line-like aperture of the second collimator can be positioned at the point where the particle beam with the line-like cross-sectional profile is to be incident through the second collimator, i.e. at the wanted position of the particle beam.

In particular, when the controller for varying the relative position between the particle beam and the target volume to be irradiated changes the position of the particle beam, the position of the line-like aperture of the second collimator can be positioned accordingly, in order to enable the particle beam to pass through the second collimator with each trajectory of the particle beam.

The irradiation device according to the invention comprises:

at least one particle beam application device,

at least one source for generating particles,

at least one acceleration device disposed upstream of the particle beam application device for accelerating the particles and for producing the particle beam from the accelerated particles.

This makes it possible to provide a complete system for irradiating a target volume to be irradiated, with which the particle beam can be both generated and made available, but with which the particle beam can also be shaped as described and directed at the target volume.

An irradiation device of this kind usually also comprises a positioning device for the target volume.

With an irradiation device of this kind, a relative position of a moving target volume and the particle beam can in particular be adjusted such that a primary movement direction of the target volume runs parallel with the line-like cross-sectional profile of the particle beam. This can be achieved in a simple manner, for example, by appropriate positioning of the target volume using the positioning device, resulting in particularly low susceptibility to over- or underdosing when irradiating a moving target volume.

The particle beam application device can in particular have additional elements for shaping the beam quality or rather the beam properties, such as e.g. a depth modulation device for controlling the particle beam energy. With a depth modulation device of this kind, the penetration depth of the particle beam in the target volume can be modulated. To manipulate the particle beam energy a so-called modulator wheel or a so-called ridge filter, for example, can be used.

The inventive method for guiding a particle beam comprises the following steps:

forming a cross-sectional profile of a particle beam such that the particle beam has a line-like cross-sectional profile,

changing a trajectory of the particle beam in a direction running perpendicular to the beam trajectory direction of the particle beam and in particular perpendicular to the line-like trajectory of the line-like cross-sectional profile.

In particular, the trajectory of the particle beam can be changed in a variable manner.

In one embodiment, for shaping the cross-sectional profile of the particle beam an oscillating magnetic field is applied to the particle beam which has a point-like cross-sectional profile, causing the particle beam to be deflected in an oscillating manner in a direction running essentially perpendicular to be beam trajectory direction. In this way, a particle beam with a line-like cross-sectional profile is produced from a particle beam with a point-like cross-sectional profile. In particular, the offset, amplitude and/or frequency of the oscillating magnetic field can be variably adjusted.

In another embodiment, the cross-sectional profile of the particle beam can be shaped by passing the particle beam through a collimator with a line-like aperture. In particular, the particle beam is first spread in a conical manner.

In one embodiment, the trajectory of the particle beam can be changed by varying the position of the line-like aperture of the collimator relative to the beam trajectory, in particular perpendicular to the beam trajectory.

In another embodiment, the trajectory of the particle beam can be changed by applying its magnetic field perpendicular to the trajectory direction of the particle beam, causing the trajectory of the particle beam to change. By varying the magnetic field, the trajectory of the particle beam can be variably modified so that e.g. scanning is made possible.

After shaping and changing of the trajectory of the particle beam, the particle beam can be de-scattered by passing the particle beam through another second magnetic field and deflecting it from its direction. In particular, after it has been de-scattered, the particle beam can be re-shaped in respect of its cross-sectional profile, in particular by a collimator.

### BRIEF DESCRIPTION OF THE DRAWINGS

Embodiments of the present invention with advantageous further developments according to the features of the sub-claims will now be explained in greater detail with reference, but not in a limiting sense, to the following drawings in which:

FIG. 1 shows a schematic overview of the design of a particle therapy system,

FIG. 2 shows a schematic overview of the design of a particle beam application device,

FIG. 3 shows a schematic overview of a particle beam application device having a collimator with a line-like aperture,

FIG. 4 shows a schematic overview of a particle beam application device with which scanning of a particle beam with a line-like cross-sectional profile [over a target volume] is effected by movement of the target volume,

FIG. 5 shows a schematic overview of a particle beam application device having a magnetic system and an electromagnetic deflecting device for shaping and deflecting the particle beam,

FIG. 6 shows a plan view onto a particle beam application device in which the shaped particle beam is de-scattered using a dipole magnet, and

FIG. 7 shows a schematic overview of individual steps.

### DETAILED DESCRIPTION OF THE INVENTION

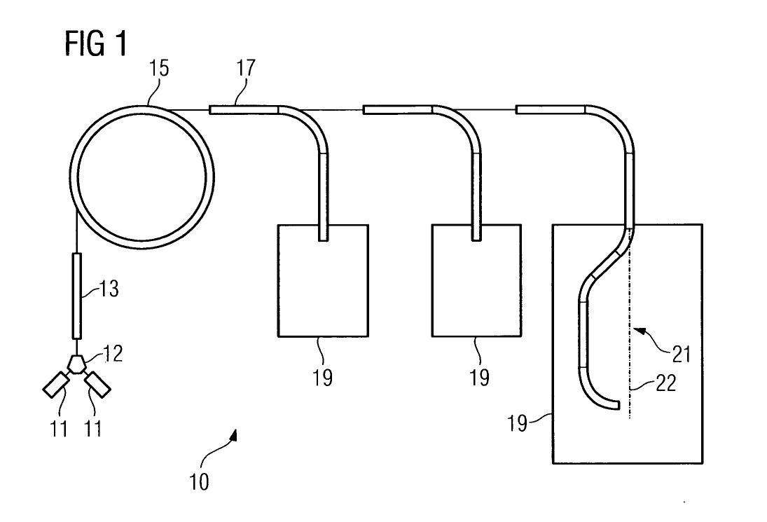

FIG. 1 shows a schematic overview of the design of a particle therapy system 10. A particle therapy system 10 is used in particular to irradiate a body, in particular a tumorous tissue, with a particle beam.

Primarily, ions such as protons, pions, helium ions, carbon ions or other types of ion are used as particles. Particles of this kind are usually generated in a particle source 11. If, as shown in FIG. 1, two particle sources 11 are present which produce two different types of ion, it is possible to switch between these two types of ion within a brief time interval. For this purpose e.g. a switching magnet 12 is used which is disposed between the ion sources 11 on the one hand and a pre-accelerator 13 on the other, thereby enabling, for example, the particle therapy system 10 to be simultaneously operated with protons and with carbon ions.

The ions produced by the ion sources 11 and where applicable selected using the switching magnet 12 are accelerated to a first energy level in the pre-accelerator 13. The pre-accelerator 13 is e.g. a linear accelerator (LINAC). The particles are then injected into an accelerator 15, such as a synchrotron or cyclotron. In the accelerator 15 they are accelerated to high energies of the kind required for irradiation. When the particles have left the accelerator 15, a high-energy beam transport system 17 guides the particle beam to one or more irradiation chambers 19. In an irradiation chamber 19, the accelerated particles are directed at a body to be irradiated. Depending on the design, this takes place from a fixed direction (in what are known as fixed beam chambers) or else from different directions via a gantry 21 rotatable about an axis 22.

The basic design of a particle therapy system 10 as shown in FIG. 1 is typical of many particle therapy systems, but may also differ therefrom; for example, depending on the acceleration of the particles, an irradiation device need not be set up as a particle therapy system.

The exemplary embodiments described below can be used both in conjunction with the particle therapy system shown with reference to FIG. 1 and in conjunction with other particle therapy systems or radiation therapy systems.

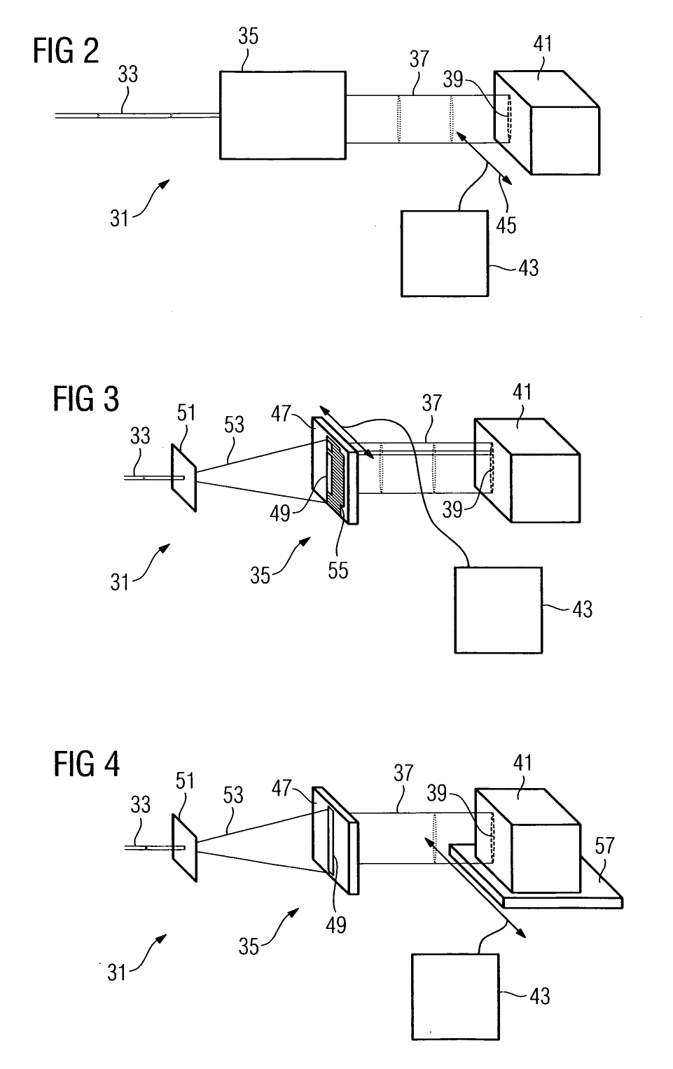

FIG. 2 schematically illustrates the design of a particle beam application device 31. An incoming particle beam 33—usually a particle beam with a point-like cross-sectional profile—enters the particle beam application device 31. The particle beam application device 31 comprises a beam shaping device 35 with which the entering particle beam 33 is changed and shaped in such a way that a shaped particle beam 37 leaving the beam shaping device 35 has a line-like cross-sectional profile 39.

The line-like cross-sectional profile 39 is characterized in that the extent of the particle beam is much smaller in one dimension than the extent of the particle beam in another dimension perpendicular thereto. For example, the extent of the particle beam in one dimension may be a few millimeters, e.g. 1 mm, 2 mm or even 3 mm or somewhat more, while the extent of the particle beam in the other dimension is in the order of centimeters, i.e. more than a few centimeters, e.g. greater than 10 cm, in particular greater than 20 cm and most particularly greater than 30 cm, up to 50 cm or more.

The shaped particle beam 37 is then directed at a target volume 41 to be irradiated. In order to be able to irradiate the entire target volume 41, the shaped particle beam 37 with the line-like cross-sectional profile 39 is scanned over the target volume 41. This means that the shaped particle beam 37 releases its energy successively at adjacent locations in the target volume 41. For this purpose the particle beam application device 31 has a controller 43 for varying the relative position between the particle beam 37 and the target volume 41. This varying of the relative position is symbolized in FIG. 2 by a double-headed arrow 45.

With reference to the subsequent FIGS. 3 to 6, various particle beam application devices 31 will now be explained in which the beam shaping device 35 and/or the controller 43 for varying the relative position between the particle beam 37 and the target volume 41 are of different design in each case.

FIG. 3 shows a beam shaping device 35 comprising a collimator 47 with a line-like aperture 49. A beam spreader 51 is disposed in the beam trajectory upstream of said collimator 47. An entrant particle beam 33 incident on the beam spreader 51 is spread in a cone-shaped manner. Passage of the conically spread particle beam 53 through the line-like aperture 49 of the collimator 47 causes the particle beam to acquire a line-like cross-sectional profile 39.

The collimator 47 shown here has an adjusting device 55 for varying the geometry of the line-like aperture 49 of the collimator 47. Shown here is an adjusting device 55 having a plurality of teeth which can be pushed into the line-like aperture 49 of the collimator 47. Said teeth can be used e.g. to limit and variably adjust the length of the line-like aperture 49 of the collimator 47. However, these teeth can also be used to interrupt the line-like aperture 49 of the collimator 47 centrally so that the shaped particle beam 37 has a discontinuous line-like cross-sectional profile 39. This provides a simple means of also irradiating target volumes “with a hole”, i.e. target volumes having a toroidal shape.

In particular, when the particle beam 37 with the line-like cross-sectional profile 39 is scanned over a target volume 41, the adjusting device 55 can be used to change the geometry of the line-like aperture 49 of the collimator 47. The changes can be controlled and implemented e.g. by means of an irradiation plan in which the geometry of the target volume 41 is stored.

In the example shown in FIG. 3, scanning is performed by displaceably disposing the line-like aperture 49 of the collimator 47 perpendicular to the beam trajectory of the particle beam. This can be done e.g. by slidably mounting the entire collimator 47 perpendicular to the beam trajectory of the particle beam, the position of the line-like aperture 49 of the collimator 47 in the beam trajectory being controlled by the controller 43 for varying the relative position. The controller 43 can also be used simultaneously to control the adjusting device 55 for varying the geometry of the line-like aperture 49.

FIG. 4 shows a similar beam shaping device 35 to that in FIG. 3. For the sake of clarity, the adjusting device for varying the geometry of the line-like aperture 49 is not shown. In contrast to FIG. 3, with the particle beam application device 31 shown in FIG. 4 the shaped particle beam 37 can be scanned over the target volume 41 by virtue of the fact that the target volume 41 is movably mounted via a positioning device 57 during an irradiation run. By e.g. continuously moving the target volume 41, the shaped particle beam 37 with the line-like cross-sectional profile 39 scans over the target volume 41.

For this purpose the controller 43 for varying the relative position between particle beam and target volume can be implemented such that it can be used to output a control signal for the positioning device 57 for the target volume 41. The positioning device 57 can in particular be a component of the particle beam application device 31.

FIG. 5 shows another variant of the particle beam application device 31.

Here the beam shaping device 35 comprises a magnetic system 59, which can be used to generate an oscillating magnetic field. A particle beam 33 with a point-like cross-sectional profile entering this magnetic system 59 can be rapidly deflected in an oscillating manner in a direction running essentially perpendicular to the beam direction. The rapid oscillation produces a shaped particle beam 37 from the magnetic system 59, said particle beam 37 essentially having a line-like cross-sectional profile 39.

The magnetic system 59 is controlled by a magnetic system controller 61 with which e.g. an offset and/or an amplitude and/or a frequency of the oscillating magnetic field can be adjusted. In particular, these parameters can be variably adjusted during an irradiation run, thereby enabling the shaped particle beam 37 to be precisely adapted to the shape of the target volume 41 during a scan.

The particle beam application device 31 additionally has an electromagnetic deflecting device 63. Said deflecting device 63 is disposed downstream of the magnetic system 59 in the beam direction. A shaped particle beam 37 passing through the deflecting device 63 is deflected from its zero position by the magnetic field generated by the deflecting device 63. The shaped particle beam 37 can be scanned over the target volume 41 by varying the magnetic field of the deflecting device 63. The electromagnetic deflecting device 63 is controlled by the controller 43 for varying the relative position.

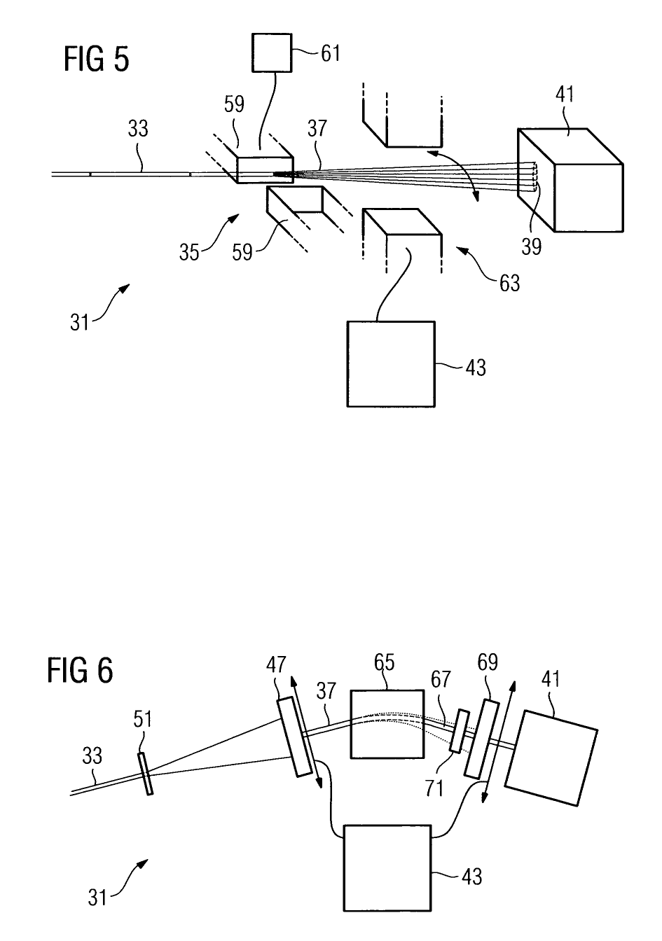

FIG. 6 shows a plan view of an advantageous further development of the particle beam application device 31 with which a particle beam guided by the particle beam application device 31 can be de-scattered.

In particular, if the particle beam with the line-like cross-sectional profile is produced by a beam spreader 51 for spreading the entrant particle beam 33 and a collimator 47 with a line-like aperture 49, scattered radiation is produced. This scatter “contaminates” the particle beam which now has particles having an undesirable mass-to-charge ratio or whose energy is at variance with the wanted energy. The shaped particle beam 37 is now guided by a dipole magnet 65 and deflected by said dipole magnet 65, the unwanted particles of the shaped particle beam 37 undergoing different deflection from that of the wanted particles of the particle beam. A particle beam 67 emerging from the dipole magnet 65 therefore consists of particles whose energy or whose mass-to-charge ratio is much more uniform than the scatter-contaminated shaped particle beam 37 entering the dipole magnet 65.

Downstream of the dipole magnet 65 can be disposed a second collimator 69 with a line-like aperture through which the emergent particle beam 67 passes, the second collimator 69 being positioned such that particles with the desired energy or rather with the desired mass-to-charge ratio can pass through the line-like aperture of the second collimator 69. This provides another filtering of particles having an undesirable energy or mass-to-charge ratio. Such particles have actually been differently deflected by the dipole magnet 65 so that they do not pass through the line-like aperture of the second collimator 69.

In particular, when the line-like aperture of the first collimator 47 is moved in the particle beam e.g. during an irradiation run, the line-like aperture of the second collimator 69 moves accordingly in the beam trajectory. The harmonized movement of the two collimators relative to one another can be controlled by the controller 43 for varying the relative position.

FIG. 6 additionally shows other optional components of the particle beam application device. Disposed in the beam trajectory is a depth modulation device 71 for controlling the particle beam energy. A depth modulation device 71 of this kind can be used to modulate the energy of the particles of the particle beam and therefore the penetration depth of the particle beam in the target volume. To manipulate the particle beam energy, a so-called modulator wheel or a so-called ridge filter can be used, for example. Alternatively, it is possible to adjust the energy of the particles of the particle beam e.g. by controlling the accelerator with which the particles are accelerated. In FIG. 6 the depth modulation device 71 is disposed downstream of the dipole magnet 65 and upstream of the second collimator 69. However, other arrangements of the depth modulation device are also conceivable, e.g. downstream of the second collimator 69 or even elsewhere.

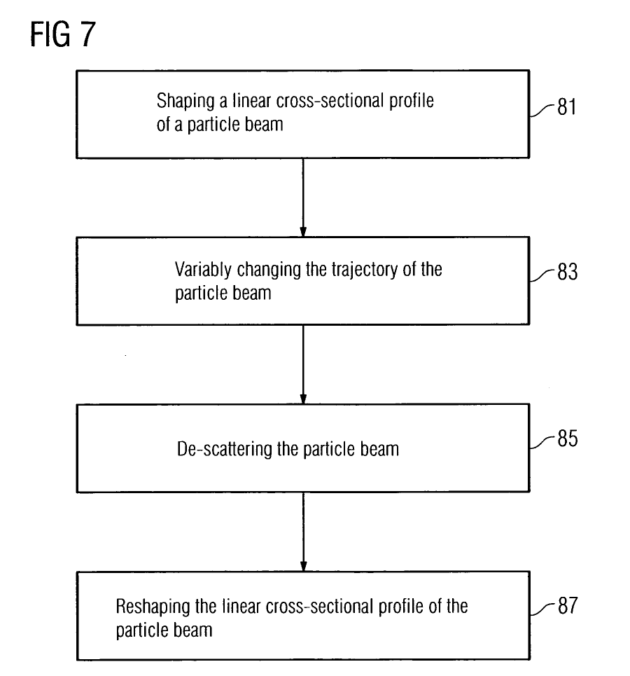

FIG. 7 shows a schematic overview of the steps in one embodiment of the method for guiding a particle beam.

In a first step 81 the cross-sectional profile of a particle beam is shaped such that the particle beam has a line-like cross-sectional profile. For this purpose the particle beam can first be fanned out and then its cross-sectional profile formed. In a second step 83 the trajectory of the particle beam is variably changed in a direction running perpendicular to the beam direction of the particle beam and in particular perpendicular to the line-like shape of the line-like cross-sectional profile. By means of the variable changing, the particle beam is guided such that the particle beam is directed in different directions, thereby enabling e.g. scanning to be performed.

After shaping and changing of the trajectory of the particle beam, the particle beam can be de-scattered in an optional third step 85 by guiding the particle beam through another second magnetic field and deflecting it in respect of its direction. In particular, after de-scattering, the particle beam can be shaped once more in respect of its cross-sectional profile in an optional fourth step 87, in particular by a collimator. A particle beam shaped and/or guided in this way can be used for various purposes, e.g. in research, for irradiating target volumes, etc.

1.-24. (canceled)

25. A particle beam application device for directing a particle beam entering the particle beam application device at a target volume for irradiating the target volume, comprising:
a beam shaping device disposed in a beam path of the particle beam that shapes a cross-sectional profile of the particle beam so that the particle beam comprises a line-like cross-sectional profile after the shaping; and
a controller that varies a relative position between the particle beam and the target volume during the irradiation so that the particle beam is scanned over the target volume.

26. The particle beam application device as claimed in claim 25, wherein the beam shaping device comprises a magnetic system for generating an oscillating magnetic field so that a particle beam with a point-like cross-sectional profile entering the magnetic system is deflected in a direction essentially perpendicular to a beam direction to form the line-like cross-sectional profile by oscillation.

27. The particle beam application device as claimed in claim 26, wherein the beam shaping device comprises a magnetic system controller for adjusting an offset, or an amplitude, or a frequency of the oscillating magnetic field.

28. The particle beam application device as claimed in claim 25, wherein the beam shaping device comprises a collimator with a line-like aperture disposed in the beam path.

29. The particle beam application device as claimed in claim 28, wherein the beam shaping device comprises a beam spreader disposed upstream of the collimator in the beam path for spreading the particle beam.

30. The particle beam application device as claimed in claim 28, wherein the collimator comprises an adjusting device for changing geometry of the line-like aperture of the collimator.

31. The particle beam application device as claimed in claim 28,
wherein the collimator varies a position of the line-like aperture relative to the beam path, and
wherein the position of the line-like aperture is controlled by the controller so that the particle beam is scanned over the target volume by moving the line-like aperture in the beam path.

32. The particle beam application device as claimed in claim 31, wherein the position of the line-like aperture is perpendicular to the beam path.

33. The particle beam application device as claimed in claim 25, further comprising an electromagnetic deflecting device disposed downstream of the beam shaping device in the beam path and being controlled by the controller so that the particle beam is deflected and scanned over the target volume.

34. The particle beam application device as claimed in claim 25, further comprising a positioning device of the target volume for receiving a control signal from the controller and for moving the target volume so that the particle beam is scanned over the target volume.

35. The particle beam application device as claimed in claim 25, further comprising a dipole magnet disposed downstream of the beam shaping device in the beam path for de-scattering the particle beam.

36. The particle beam application device as claimed in claim 35, further comprising a second collimator with a line-like aperture disposed downstream of the dipole magnet in the beam path.

37. The particle beam application device as claimed in claim 36, wherein the second collimator varies a position of the line-like aperture of the second collimator relative to the beam path.

38. The particle beam application device as claimed in claim 35, wherein the cross-sectional profile of the particle beam is reshaped after being de-scattered.

39. The particle beam application device as claimed in claim 25, further comprising a depth modulation device for modulating a penetration depth of the particle beam.

40. An irradiation device for irradiating a target volume by a particle beam, comprising:
a source device that generates particles;
an acceleration device that accelerates the particles and generates the particle beam from the accelerated particles; and
a particle beam application device disposed downstream of the acceleration device that directs the particle beam entering the particle beam application device at the target volume, the particle beam application device comprising:
a beam shaping device disposed in a beam path of the particle beam that shapes a cross-sectional profile of the particle beam so that the particle beam comprises a line-like cross-sectional profile after the shaping, and
a controller that varies a relative position between the particle beam and the target volume during the irradiation so that the particle beam is scanned over the target volume.

41. The irradiation device as claimed in claim 40, wherein the relative position between the particle beam and the target volume is adjusted so that a primary movement direction of the target volume is parallel to the line-like cross-sectional profile of the particle beam.

42. A method for directing a particle beam at a target volume, comprising:
shaping a cross-sectional profile of the particle beam so that the particle beam comprises a line-like cross-sectional profile after the shaping;
changing a beam path of the particle beam in a direction perpendicular to a beam direction of the particle beam and perpendicular to the line-like cross-sectional profile of the particle beam; and
irradiating the target volume by the particle beam.

43. The method as claimed in claim 42, wherein the beam path of the particle beam is changed by a magnetic field perpendicular to the direction of the particle beam.
